(19) United States
(12) Patent Application Publication
(10) Pub. No.: US 2021/0228331 A1
(43) Pub. Date: Jul. 29, 2021

(54) SURGICAL TOOLS AND KITS FOR TENDON OR LIGAMENT REPAIR USING PLACENTAL, AMNIOTIC, OR SIMILAR MEMBRANES

(71) Applicant: Michael A. Scarpone, Bloomingdale, OH (US)

(72) Inventor: Michael A. Scarpone, Bloomingdale, OH (US)

(21) Appl. No.: 15/482,711

(22) Filed: Apr. 7, 2017

Related U.S. Application Data
(60) Provisional application No. 62/319,783, filed on Apr. 7, 2016.

(51) Int. Cl. A61F 2/08

(52) U.S. Cl. CPC A61F 2/0811; A61F 2230/0069; A61F 2002/0817

(57) ABSTRACT

Sock-like and “button”-type surgical implants, as well as kits containing such devices for use by a surgeon during an operation, are disclosed for surgically reattaching a tendon or ligament to a bone. In sock-like implants, a “toe” portion (or “distal tip”) will receive and hold a bone dowel, which can be pressed into a hole drilled into a bone surface, to anchor and hold the implant in position. “Button” implants can use a “nipple” component to hold a bone dowel anchor. The remainder of either type of implant will contain specialized repair cells, including platelets and “stromal precursor cells”. These implants can be made from placental membranes, treated-collagen membranes or meshes, or other biological materials, to further enhance their ability to stimulate the reattachment of damaged tendons and ligaments to bones.

### RELATED APPLICATION

This application claims priority under 35 USC 119(e) based on provisional application 62/319,783, filed on Apr. 7, 2016.

### BACKGROUND

This invention is in the field of disposable supplies and equipment for use during specialized types of orthopedic surgery and “sports medicine”.

Extensive work has been done by surgeons and researchers to develop ways to treat and repair damaged tendons and ligaments. As a general rule to distinguish between those two terms, a tendon is coupled directly to muscle tissue; it attaches a muscle to a bone, or to some other structure. By contrast, a ligament does not directly involve muscle tissue; instead, ligaments attach non-muscle tissues (such as bones, cartilage, etc.) to other non-muscle tissues.

Except for that functional distinction, tendon and ligament tissues are very similar to each other. Both are composed of flexible but non-stretchable and exceptionally strong bundles of collagen, the fibrous protein that forms the extra-cellular “matrix” which holds essentially all soft tissues together, in mammals and other vertebrates. Those bundles of extra-cellular fibrous proteins could not and would not be as strong as they are, and could not function as well as they do, if they had large numbers of living cells interspersed among the fibers, since cells are covered by soft outer membranes which simply cannot resist substantial tensile forces; therefore, if large numbers of cells were dispersed and distributed throughout the bundles of fibrous proteins that form tendons and ligaments, those cells would seriously disrupt the tight alignment and dense packing of those fibers, in ways which would seriously degrade and impair the strengths of the collagen bundles. As a result, tendons and ligaments do not have cell densities that even remotely approach the cell densities in muscles or internal organs; and, since they do not need to support the metabolic needs of (or metabolite removal from) large numbers of living cells, tendons and ligaments have abnormally low levels of circulating blood supply, compared to muscle and organ tissues.

As a result of those factors, if a tendon or ligament becomes damaged, due to injury, infection, or disease, it will have only a very limited (and in most cases inadequate) ability to regrow, regenerate, or repair itself. Accordingly, if tendons and ligaments become injured or diseased beyond the level of a minor or moderate sprain, they usually require medical or surgical intervention of some sort, if the patient can afford it and has access to such treatment.

Such treatments generally can be divided into two major categories. The first (and older) category involves mechanical-type interventions, which include:

(1) the use of sutures, pins, staples, or similar attachment devices, to reattach one end of a tendon or ligament to the bone or other structure that it became separated from; and/or,

(2) the use of intact tissue grafts, taken from elsewhere in a patient's body, or, in some cases, using tendon or ligament grafts from cadavers. Tendon and ligament grafts from cadavers which can usually be done without actively triggering an immune rejection response, since tendon or ligament grafts involve very few cells, and mostly just contain bundles of protein molecules which are very nearly identical to each other, not just in all humans, but in all mammals.

The second and newer category involves biological approaches that are intended to enable and stimulate the regeneration of new tendon or ligament tissue, by using transplanted cell types that have been specifically selected for such use, because they are capable of maturing into cells which can actively generate and secrete the types of collagen fibers that can be assembled into new and/or replacement tendon or ligament tissue.

One particular method of cell harvesting that has been found to be exceptionally useful for tendon or ligament regeneration involves the use of specialized devices called “trocar” devices, which are well known to orthopedic surgeons. Most trocar devices contain a combination of:

(1) a rigid shaft, made of stainless steel or a similar hard and strong biocompatible alloy, in the shape of a long and narrow hollow sleeve or cylinder, with a diameter that is larger than most types of conventional hypodermic needles;

(2) an internal rod with a very sharp tip, made of a strong alloy that can be pushed and driven directly into a large and heavy bone such as a pelvis, and which can then be withdrawn from the hollow tube of the sleeve/cylinder, in a manner which will then allow the hollow sleeve/cylinder to be pushed deeper into the relatively soft “marrow” tissue that resides inside the hard outer layer of bone that surrounds pelvic, femoral, and other large bones; and,

(3) a handle with several specialized triggers or other manipulators, which allow certain types of specialized operations to be performed by the surgeon who is handling and controlling the trocar.

If a trocar is driven through the entire outer surface of a pelvic bone at a conventional location site, until it reaches the marrow tissue inside the bone, the bone dowel usually will be about 1.5 to about 2 cm in length. It will contain “cortex bone” (from the hard outer surface of the pelvic bone) at one end of the dowel, and “spongy bone” (from the layer where the dense bone undergoes a non-planar transition, which forms an interface between the hard bone, and the internal bone marrow) at other end of the dowel. The bone dowel usually will have a diameter somewhere between about 1/12th to 1/18th of an inch, depending on whether a 12-gauge, 14-gauge, 16-gauge, or 18-gauge trocar was used to harvest it.

After the sharp tip of a trocar cylinder has penetrated all the way through the outer bone surface, and has reached the marrow tissue, a suction device (also called an “aspirator”) can then be used to suction a desired quantity of marrow tissue out of the interior of the pelvis. That tissue (sometimes called an “aspirate”, in medical terms, since it was obtained via the process which doctors call “aspiration”) will emerge in a semi-liquefied form, which can be manipulated and shaped in a manner similar to toothpaste or clay.

Both the bone dowel, and the marrow aspirate (or a targeted set of cells obtained from the marrow aspirate) will be used as described below (in the description of the invention) to help repair a tear or other injury in a tendon or ligament.

In the prior art, various researchers and companies have tested and used various types of membranes to help hold marrow aspirate cells in place, at a repair site where a tendon or ligament is being repaired. In most such cases, that type of repair effort involves injecting marrow aspirate cells directly into a tear or other damage site in a tendon or ligament, and then securing a membrane (such as a placental or amniotic membrane, or a membrane made from processed collagen obtained from cow tendons) on top of the tendon or ligament surface where the marrow cells were injected. In this approach, the membrane segment placed over the injection site is used to help hold the injected marrow aspirate cells in or near the site of the injection, in the hope that if they remain at that injection site for a longer period of time, they will be able to do more to help repair the injured tendon or ligament, by generating new collagen fibers that can and will be incorporated into the injured tendon or ligament at the damage site.

This current invention takes a different approach, as described below.

The description below uses, as an illustrative example, a “rotator cuff” repair in a shoulder joint. As is well known to orthopedic surgeons, rotator cuff injuries are among the most common and frequent tendon or ligament injuries that occur in athletes, and in other people from all walks of life.

As illustrated in numerous sources which can be quickly located by a Google image search for “rotator cuff”, the “rotator cuff” in a shoulder joint is formed by four distinct tendons, which connect four different muscles to the “humeral head”. The “humerus” is the long bone inside the upper arm, which extends from the elbow up to the shoulder. Its upper end has an enlarged convex rounded surface, called the “humeral head”, which generally interacts with a shallow concave “socket” (usually called the glenoid socket or cavity) in a “scapula” bone (i.e., the “shoulder blade” bone; the glenoid socket is part of the shoulder blade, located near the “upper outer corner” of the shoulder blade). Unlike other ball-and-socket joints (such as in the hips), the interface between the “humeral head” and the “glenoid socket” can be referred to as “shallow and loose”, in a manner which gives the arms of humans (and apes) a substantially greater range of motion than the legs have, relative to the pelvis).

As a brief digression, it can even be noted that the different types of shoulder joints that evolved and emerged, when larger and heavier apes veered off into their own separate branch within the larger family of monkeys, became the definitive anatomical difference that distinguishes “apes” from “monkeys”. Shoulder joints play crucial roles in how monkeys and apes gather food. Monkeys, which generally are lighter and more nimble than apes, will walk on top of a branch to reach a piece of fruit growing in a tree. By contrast, an ape will hang down below the branch, using its powerful arms to hold on to and hang down from the branch, since that type of approach to an item of food places the ape both: (i) closer to the ground, and (ii) in a better position to survive a fall, if the branch breaks. Apes can readily hang down below branches, because their shoulder joints are more flexible, and allow greater ranges of motions, compared to the shoulder joints of monkeys.

But, alas, that greater freedom and range of motion came with a price, because the shoulder joints of humans often create and contribute to serious joint problems, most of which involve “rotator cuffs.”

As mentioned above, four distinct tendons connect four different muscles to the rounded “head” at the upper end of the humerus bone in the upper arm. The uppermost (and most exposed, and vulnerable) of those four tendons (i.e., nearest the top surface of the shoulder joint) is called the “supra-spinatus.” In the most frequent and common type of rotator cuff injury, the tendon which attaches the supra-spinatus muscle to the humeral bone becomes partially detached, in a manner which seriously weakens the joint, and causes a range of pain that increases from mild and tolerable levels, when the arm is kept still and pointing downward, up to severe and even excruciating levels, if the person tries to raise his arm vertically.

Currently, there are no “ideal” ways to repair a “torn” rotator cuff, which in most cases involves trying to reattach the end of a tendon, to a bone. Every method in use today has its limitations and shortcomings.

The same also applies to any other type of tendon or ligament repair. This notably includes the repair of tendons and ligaments in the knees, such as the well-known “anterior cruciate ligament” (ACL) in the knee, the ligament that is most commonly injured when athletes suffer knee injuries.

As a final item of background information, a number of comments have been made, in a separate patent application (Ser. No. 15/482,696, by the same inventor herein) about “placental” membranes. These include membranes obtained from a placenta, which is the complete set of “interface” tissues that enable an embryo/fetus/baby to grow inside a womb. The placenta normally is expelled by the female's body soon after she has given birth to a baby, and the complete set of placental membranes include: (i) membranes obtained from the amniotic sac, and (ii) membranes obtained from the umbilical cord. For a number of reasons, these types of membranes have become of substantial interest in the medical field, and they are being actively used to treat various types of soft tissue injuries. Accordingly, the above-referenced patent application should also be consulted for additional background information about those types of membranes.

Alternately or additionally, more extensive background information about such membranes can be obtained by consulting the websites of numerous companies which sell such membranes, including Skye Biologics Inc. (skyebiologics.com); Amniox Medical Inc. (amnioxmedical.com); MiMedx Group Inc. (mimedx.com); Alliqua BioMedical Inc. (alliqua.com); Osiris Therapeutics Inc. (osiris.com); Applied Biologics Inc. (appliedbiologics.com), Burst Biologics LLC (burstbiologics.com), and BioD LLC (biodlogics.com). In addition, articles have begun to appear in the medical literarure, including two recent review articles (Riboh et al 2016, and McIntyre et al 2017), which review and describe various reports on early efforts to test and use placental membranes to repair tendon or ligament injuries in adults.

Accordingly, one object of this invention is to disclose and teach what is believed to be an important advance, and improvement, in the methods and devices that can be used to treat tendon and ligament injuries or other damage.

Another object of this invention is to disclose and teach a composition of matter and/or “article of manufacture”, which comprises a specific combination of materials which will be created by a surgeon who is performing this type of tendon or ligament repair, during the course of a surgical repair procedure.

A third object of this invention is to disclose and teach an article of manufacture, which comprises a new type of surgical device which can be manufactured, packaged, and shipped under sterile conditions, and which will enable surgeons to perform tendon or ligament repairs more rapidly and effectively than could ever previously be achieved.

These and other objects of the invention will become more apparent through the following summary and description.

### SUMMARY OF THE INVENTION

Sock-like surgical implant devices, and “button”-type surgical implants, as well as sealed sterile kits containing such devices, to be opened and used by a surgeon during a tendon or ligament repair operation, are disclosed, which are optimized for use in surgical repairs of tendons or ligaments.

The “toe” portion (which can also be called a distal tip, or similar terms) of these sock-like device(s) will be sized and designed to receive and hold at least a portion of a bone dowel. The bone dowel segment which will enter the sock-like implant device first should include a segment of hard cortex bone, so that after that hard bony segment has reached and settled into the tip of the “toe” portion, that toe portion can be forcibly pressed into an accommodating hole which has been drilled into a bone structure, to anchor and hold the sock device (and its contents) in position.

The remainder of the sock-like implant device will be designed to hold a quantity of cells which will actively secrete collagen fibers of a type which will help regenerate, repair, and/or re-attach a torn, detached, or otherwise injured or damaged segment of tendon or ligament.

Accordingly, by inserting a hard bony segment into a closed “toe” end of a sock-like implant, and by driving that bony segment into an accommodating hole that has been drilled into a bone, the cell preparation can be held inside the remaining portion of the sock, to ensure that the cells stay in a location where they will do the most good, in helping to reattach or otherwise repair a damaged segment of tendon or ligament.

Furthermore, these types of sock-like implants can be made from placental membranes, treated-collagen membranes, or other biologically-derived membranes or membrane-like materials (including processed collagen meshes, also described herein) to further enhance their ability to stimulate the regeneration, repair, and/or reattachment of damaged tendons and ligaments.

### BRIEF DESCRIPTION OF THE DRAWINGS

FIG. 1 is a side (elevation) cutaway view of a miniaturized sock-like implant device, in the shape of a generally cylindrical “tube sock”, made from a placental or processed collage membrane. This drawing indicates the zones that will be filled by: (i) a dowel segment, made of hard “cortex” bone, (ii) “spongy” bone, of the type that forms the interface between hard bone and bone marrow, in a pelvic bone; and, (iii) aspirated cells from bone marrow.

FIG. 2 is a side (elevation) cutaway view of a sock-like implant device, in which the anchoring segment (filled by hard cortex bone) is coupled to an enlarged chamber which can be sculpted and shaped by a surgeon into any desired shape, such as a flattened disc-like shape which will immobilize a set of aspirated marrow cells at the interface between a bone surface, and the “underside” surface of a tendon or ligament which must be reattached to the bone.

FIG. 3 is a side (elevation) cutaway view of a sock-like implant device, in which the anchoring segment (filled by hard cortex bone) is coupled to a first enlarged chamber, which will hold a first set of aspirated bone marrow cells, and a second enlarged chamber, which will hold a second set of aspirated bone marrow cells, thereby allowing two distinct batches of aspirated marrow cells to be held in position at both the “underside” surface of a tendon or ligament (i.e., at the interface where the tendon or ligament will be reattached to the bone, and on the “outer” surface of the same tendon or ligament.

FIG. 4 is a perspective view of a “button implant”, made from a processed collagen mesh which can have any desired levels of density, porosity, and permeability, and which can hold cells which have been embedded into it (such as through centrifugation), having a “finger” protrusion that will hold a bone dowel, to help securely anchor the implant to a bone surface, in a location which will hold the cells (plus any hormones, growth factors, etc.) in location where a tendon or ligament needs to be reattached to a bone surface.

FIG. 5 depicts a surgical kit, with several useful components (including a set of dilator tubes, a set of tamping devices, and a supply of biological membranes or collagen meshes) sealed inside sterile envelopes.

### DETAILED DESCRIPTION

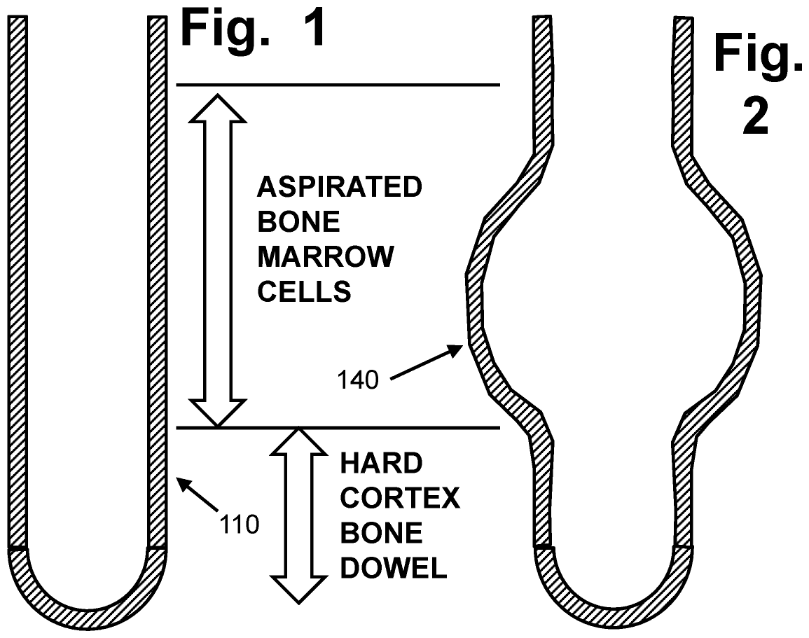

The initial discussion below will focus upon sock-type implants, as illustrated in FIGS. 1-3; following that discussion, “button implants” made of collagen meshes will be disclosed.

In one embodiment of a sock-type implant, shown as implant 110 in FIG. 1, the implant device comprises a structure that is analogous to a miniaturized “tube sock” (i.e., a generally cylindrical piece of flexible fabric-type material which has one closed end, and one open end). This generally cylindrical design can allow maximal contact of the cells contained within that cylinder, with the entire thickness of a tendon or ligament segment through which it is passed, by the surgeon. Accordingly, this design is suited for use in repairing tendons or ligaments at locations which have substantial thickness.

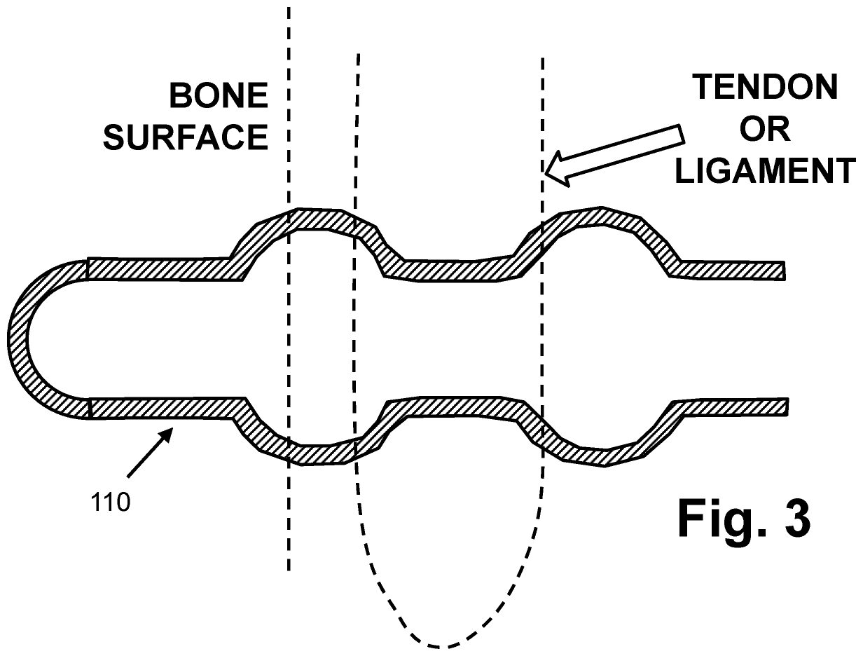

An alternate shape can be used, if desired, as depicted by implant 120 in FIG. 2, to increase the number and quantity of transplanted cells which are placed directly in contact with: (i) either or both sides of a segment of tendon or ligament, and/or (ii) an interface area where a segment of tendon or ligament needs to be reattached to a bone or other internal structure. For example, a sock-like implant device as described herein can have, positioned next to the cylindrical “toe portion” which will hold the hard cortex bone segment for anchoring, a bladder-type or disc-type “chamber” shape having an enlarge diameter, to hold a greater quantity of cells from the bone marrow aspirate, so that more of those cells will be at the location where a segment of tendon or ligament that is being reattached to another structure.

Another alternate shape can be created and used, as depicted by implant device 130 in FIG. 3, having two such enlarged chambers. A surgeon can pack a first quantity of marrow cells into the first enlarged chamber which is adjacent to the “toe” portion and the bone dowel, prior to pressing the damaged tendon or ligament segment against that quantity of marrow cells. The surgeon will then pin, staple, suture, or otherwise secure the tendon or ligament segment to the bone or other structure. Once that securing step has been completed, the surgeon can then load a second quantity of marrow cells into the second enlarged chamber of the sock-like implant, so that the second quantity of marrow cells will be pressed directly against a second surface (which in most cases will be an outer surface) of the tendon or ligament segment which is being repaired or reattached. This design can allow not just one but two different sets of stem cells to begin secreting collagen fibers which can help regenerate new tissue, to help repair and/or reattach the damaged tendon or ligament.

These “sock-like implants” will be made of placental membranes (a category which also includes amniotic and umbilical membranes), processed collagen membranes or meshes (as described in more detail below), or other biologically-derived membranes (or combinations of such membranes) which have been shown to help promote cell growth and tendon or ligament tissue regeneration.

Regardless of any specific design that may be chosen, the “toe” (or distal tip, or similar terms) of the sock-like device will be sized to receive and hold:

(1) a “bone dowel”, ranging from about 1/12th to about 1/18th of an inch in diameter, and up to about 2 cm in length, that has been obtained from a pelvic or other large bone, and which preferably should include both hard cortex bone and more porous “spongy bone”; and,

(2) a suitable quantity of a paste-like “marrow aspirate”, which will contain a large number of “stem cells” of a type which can help regenerate and repair tendon or ligament tissue, if injected into an injury site in a tendon or ligament.

During a surgical repair procedure, the surgeon will obtain both the bone dowel, and the marrow aspirate, from the same patient who has suffered the tendon or ligament injury or damage. The hard cortex portion of the bone dowel will be inserted into the “tube sock” first, so that it will reach the distal tip (or “toe”) of the “sock”. The “distal tip” (or toe) of the “sock” which contains the bone dowel will then be inserted by the surgeon into a hole which has been drilled in a bone to which a tendon or ligament needs to be attached (or reattached, due to a tear or other injury). The surrounding bone will then grip that bone dowel in a manner which will thereafter hold it in place, either by natural means, or with the aid of cement, sutures, etc. The remainder of the “sock” will then hold the newly-inserted stem cells (obtained from the “marrow aspirate” material that was suctioned out of the pelvic bone) in place, in the midst of the injured tendon or ligament tissue. After that has been accomplished, the site of insertion of the “tube sock” containing the bone and stem cells will be covered by another segment of placental or processed collagen membrane, to further help hold the stem cells in place while they do their work over the following days and weeks.

In this manner, by using this type of “miniature tube sock” device (made of a growth-promoting placental or collagen membrane) in conjunction with a bone dowel (as an anchoring device) and marrow-derived stem cells (which can help regenerate tendon or ligament tissue), a surgeon can enable a better and faster form of tendon or tissue regeneration than could previously be achieved by any other means.

To prevent potential misunderstandings, each word in the phrase, “miniaturized sock-like surgical implant” requires a brief discussion.

“Miniaturized” indicated that it will be a fraction of an inch in diameter, rather than having a diameter comparable to an actual sock that fits on a foot. Most “trocar” tools, of the type that are used to puncture and penetrate a pelvic bone, to enable suctioning (“aspiration”) of bone marrow cells from the marrow tissue inside the pelvic bone, have diameters that range from 12 gauge (i.e., with an outside diameter of about 1/12 of an inch), to 18 gauge (i.e., with an outside diameter of about 1/18 of an inch). These sock-like membrane structures can be manufactured with clearly excess length (i.e., beyond anything that is likely to be needed in actual use), and a surgeon can simply cut down that surplus length, to any desired size. For example, at this initial stage of planning, it is presumed that a manufactured length of about 2 to 3 inches (about 7 to 9 cm) will be suitable, since it will be greater than anything which is likely to be actually needed, for reattaching the end or tip of a tendon or ligament, to a bone surface. If a surgeon needs a longer length, for some particular type of surgical procedure s/he is planning, s/he can custom-order any desired number of sock-like implants, in one or more longer lengths.

On the subject of longer lengths, it should be understood that, after these types of “miniaturized sock-like surgical implants” become commercially available, and surgeons begin using them, and seeing for themselves what they can accomplish, they are likely to be adapted for other types of surgical uses, beyond just reattaching tendons or ligaments to bone surfaces. Such uses are intended to be covered by the claims here, if they involve the manufacture and/or surgical use of miniaturized sock-like surgical implants” as disclosed and claimed herein. Accordingly, since these devices can be simply and quickly cut down, to any desired length, by a surgeon who knows what length s/he will need for any particular implantation procedure, no fixed length is going to be specified herein, for their manufactured lengths.

The term “sock-like” means that these devices will have generally cylindrical shapes, with one closed end (referred to herein as the toe, or the distal tip), and one open end. The open end will allow a surgeon to insert (or load, or similar terms) a bone dowel, and a quantity of bone marrow cells, into the device, immediately before the loaded implant (with the newly-inserted cells it is holding) is surgically inserted into the patient. The closed end will cause the “distal tip” of the bone dowel to stop, and not keep traveling farther, when it reaches that closed end of the “sock-like” device. This will enable the “toe” end of the implant, and the hard bone dowel segment which has been pushed all the way into the sock-like implant, to work together to securely anchor the loaded implant to a bone surface, by firmly pushing (with tapping and/or rocking action, or other means) the distal end of the bone dowel into an accommodating hole which has been drilled into the bone surface which will provide the anchoring attachment. If desired, that pushing, tapping, or other anchoring step can be completed, to ensure solid and secure fixation to the bone, before any bone marrow cells are loaded into the sock-like implant device.

The phrase “surgical implant” means that: (i) the implant device (as well as any bone segment and cells loaded into it) will be inserted into a patient (such as into a shoulder, knee, hip, or other articulating joint that needs repair) by a surgeon (regardless of which types or combinations of scalpels, arthroscopic tools, or other tools are used by the surgeon); and, the implant device will be left in that site, inside the patient (presumably for the entire remaining life of the patient, unless the surgeon chooses otherwise), after the surgeon has completed the procedure.

In addition, orthopedic surgeons will realize that other, additional procedures for implanting these types of implant devices can also be used. As just one example, a guide wire which shows up on an ultrasound monitor can be used to guide the proper placement of a sock-type implant having a barb, an eyelet to receive a screw, or other attachment component, to a targeted location on a bone surface where a tendon or ligament needs to be reattached. A thin cylindrical container containing a deflated balloon catheter, which is loaded with a cell preparation and/or other biological material, can then be guided to that same location, by the guide wire. The balloon catheter can then be gradually inflated as it is slowly retracted from its deepest position, so that it will force out the cells and/or other biological materials, into the interior volume of the sock-like implant device. That is just one example of a type of placement procedure that orthopedic surgeons can use.

### Semi-Synthetic Collagen Mesh Materials; Button Implants

Another line of technology merits attention, since it can provide an alternate chemical pathway for creating biologically-derived collagen-containing membranes with any desired level of porosity and permeability, for potential use as disclosed herein.

The pathway for creating these types of membranes was initially developed by Ioannis Yannas, a mechanical engineer and textile specialist at MIT, in conjunction with James Burke, a surgeon who specialized in treating burn victims. Together, they set out to develop membranes that could encourage cell growth in order to replace skin, in burn victims. Their work is described in a number of patent from the early 1980's, including U.S. Pat. No. 4,060,081 (“Multilayer membrane useful as synthetic skin” and U.S. Pat. No. 4,280,954 (“Crosslinked collagen-mucopolysaccharide composite materials”).

As a brief overview, the process they eventually settled upon involved the following series of steps:

1. Collagen fibers were obtained by processing cowhides, which are available in abundant supply from slaughterhouses;

2. The collagen fibers were combined and reacted with a class of molecules called “muco-polysaccharides” (MPSs) or “glycos-amino-glycans” (GAGs), which are naturally occurring compounds that contain a combination of sugar rings, and amine substituents, strung together in ways that create naturally-occurring polymers. Yannas and Burke found that, by adding controllable quantities of selected GAG compounds to a batch of prepared collagen fibers from cowhide, it was possible to reduce and limit, in a controllable manner, the rates at which digestion and resorption of the collagen fibers occurs (mainly due to collagenase, an enzyme which attacks and breaks down old collagen fibers, to release their amino acid building blocks, so that they can be reformed into new collagen fibers). This allowed them to create collagen-based membranes which could be designed to last for a sustained period of time (such as weeks or months, depending on the severity of the burn wounds suffered by a specific patient), but which nevertheless would eventually be resorbed completely, by the patient's body, as new skin was regenerated by the patient.

3. The resulting long-chain polymeric molecules were then reacted, in aqueous solution, with a “two-handed” crosslinking agent, such as glutaraldehyde, which has two reactive and “sticky” aldehyde groups at opposite ends of a relatively short spacer chain. The operating parameters and conditions of that cross-linking reaction could be controlled, by manipulating factors such as: (i) the quantity of glutaraldehyde which was added to the reaction mixture; (ii) the duration of the reaction/incubation period, before any unreacted glutaraldehyde was rinsed out; and (iii) the temperature, acidity, salt content, and other parameters of the cross-linking step. By adjusting and controlling those parameters, it was possible to create any desired level of cross-linking attachments, in which the “spacer chains” of the glutaraldehyde molecules formed short molecular bridges and attachments, which linked together the long collagen-plus-GAG strands.

4. When the reacted mixture was ready, it was suspended in water, and then frozen, in a relatively thin and shallow layer, to establish a fixed three-dimensional mesh-type lattice having a planar shape with a wide surface area, and having a desired level of thickness (to render it suitable for forming sheets of a skin-cell growth template which could be laid down across a burned area which needed skin regeneration, on a burn patient). The fibers within that frozen and solid preparation contained mainly naturally-occurring collagen fibers, with a small quantity of GAG molecules added, to control resorption rates, and with short cross-linking attachments (created by the glutaraldehyde reagent) holding together the long collagen fibers.

5. The frozen sheets were then lyophilized (i.e., subjected to a “freeze-drying” operation, under an intense vacuum which cause the ice molecules to “sublimate”, which means they converted from frozen ice, into vapor which was suctioned away and removed, without passing through a liquid state). By removing the water molecules in that manner, without allowing them to pass through a liquid state which would have altered and damaged the membrane that was being formed, the freeze-drying step left behind a solid but flexible membrane, made of cross-linked collagen fibers having a micro-structure which emulated the extra-cellular collagen matrix of human skin, closely enough to allow and encourage embedded skin-forming cells to reproduce, and grow to “confluence” (i.e., forming an intact and cohesive skin membrane).

6. As the final preparative step to make the membrane ready for grafting onto the skin of a burn victim, a layer of skin, several cells thick, was harvested from the patient, from some area on the patient's body which had not been burned, using a specialized tool that resembles a single-blade razor. That skin-harvesting operation was deep enough to obtain large numbers of the precursor cells which create new epidermal cells, without removing all of those precursor cells. That harvesting procedure left behind a patch of reddened and tender skin which resembled a severe sunburn, but which healed within a week. The harvested strips of tissue were treated by a digestion mixture, which released the precursor skin cells without damaging them, so that they could be suspended in a liquid carrier containing salts and nutrients which protected and sustained the cells. That cell suspension was then placed in a centrifuge basket, which contained a segment of the prepared collagen mesh, pre-positioned and laying flat in the bottom of the centrifuge basket. A brief centrifuging operation drove the cells down into the pores and interstitial spaces in the collagen mesh, thereby embedding those cells within that mesh. If desired, selected hormones and growth factors, to stimulate cell growth and reproduction, can also be added.

This created a cell-carrying collagenous membrane, somewhere at a midpoint between natural and synthetic, which was ready to be grafted onto a badly burned area, in a burn patient.

Accordingly, these same steps (or any similar or analogous but improved processes which have been developed since the 1980s, or which may be discovered in the future), for treating collagen to create porous mesh-type materials that can promote cell growth and tissue reconstruction, can be adapted and used to create sock-like implant devices as disclosed herein.

Three of the more interesting and appealing aspects of these types of materials are:

(i) these types of collagen meshes can be created with any levels of porosity and permeability that are of interest, for creating porous and permeable implant materials that can allow collagen secretion and rebuilding, and balanced biological activities which allow both cell anchoring and cell migration, by bone marrow cells or other selected cell types which can help accelerate the regeneration or reattachment of injured or damaged tendons and ligaments.

2. these types of semi-synthetic collagenous meshes can be directly bonded to entirely natural membranes (such as placental, amniotic, or umbilical membranes), by using chemical adhesives that have been developed for gluing connective tissues together. A variety of candidate adhesives have been recently disclosed which appear to offer major improvements compared to earlier-generation adhesives such as cyanoacrylate and methacrylate. These candidate adhesive compounds will immediately appear if the National Library of Medicine database is searched for “tissue adhesive” (Kelmansky et al 2017, entitled “Strong tissue glue with tunable elasticity,” offers an example of such an article). Accordingly, numerous types of tissue adhesives are known, and it would be a straightforward task, which would not require undue experimentation, to determine which ones can securely attach a placental membrane, to a processed collagen mesh as described above, while maintaining a soft and pliable interface rather than creating a hardened ridge of “dried glue”. By using a suitable adhesive to bond a placental membrane to a processed collagen mesh, an implant device can be assembled which will provide an optimal combination of: (i) the tissue-growing benefits provided by the placental membrane segment; and (ii) the desired levels of porosity, permeability, stretchability, and other controllable mechanical properties, which can be created in a processed collagen mesh.

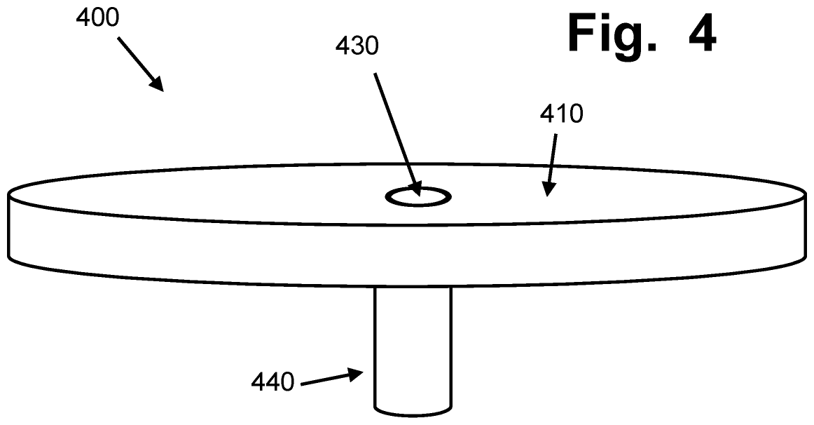

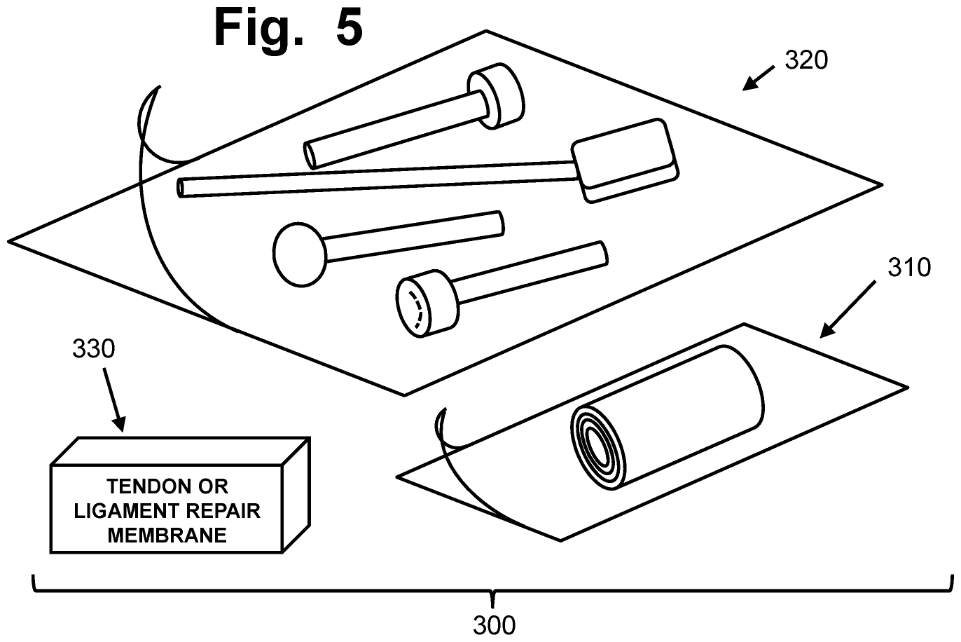

Alternately, a collagen mesh can be created which has a shape as depicted in FIG. 4. That drawing depicts a “button implant” 200, made of a processed collagen mesh. Button implant 200 has a “skirt” component 210 (which also can be called an apron, periphery, flap, or similar terms), and a “finger” component 220 (which also can be called a sleeve, protrusion, nipple, or similar terms). The skirt component 210 presumably will have a generally round or elliptical shape, to avoid any corners. It will have sufficient thickness and porosity to hold a large number of bone marrow or similar cells; however, the thickness (relative to the outer diameter) is exaggerated in the drawing, and the peripheral edge can be gradually tapered, to create a thin outer band, to avoid any hard-edged “shoulder” surfaces that might irritate or disrupt surrounding tissues. The finger component 220 will be sized to receive and hold a bone dowel segment, which will be inserted into the implant device 200 via a hole or tunnel 230 which passes through the skirt component 210. The hard cortex tip of the bone dowel segment, enclosed within the finger component 220, can be pressed into a hole that has been drilled into the bone surface, to help anchor the implant.

Accordingly, this type of device can lie flat, and nearly flush (with thin tapered outer edges), against a bone surface, when a segment of tendon or ligament is being reattached to the bone surface at that location. The processed collagen mesh will hold bone marrow cells (or other selected types of cells) embedded within the mesh material, along with hormones, growth factors, nutrients, or any other compounds which the surgeon choose to insert into the collagen mesh. It will be positioned and anchored where a reattachment needs to be made, between a tendon or ligament and a bone surface.

Alternately, this type of “button implant” made of a processed collagen mesh can be adapted for various other types of surgical repair procedures, where its placement and anchoring, as a permeable holder for certain selected types of cells, hormones, growth factors, and any other selected additives, is likely to provide a significant benefit for the patient, in the judgment of the surgeon who is treating that patient.

Thus, there has been shown and described a new and useful set of tools, devices, and articles of manufacture, for enabling improved repairs of damaged or diseased tendon and ligament segments. Although this invention has been exemplified for purposes of illustration and description by reference to certain specific embodiments, it will be apparent to those skilled in the art that various modifications, alterations, and equivalents of the illustrated examples are possible. Any such changes which derive directly from the teachings herein, and which do not depart from the spirit and scope of the invention, are deemed to be covered by this invention.

1. A surgical implant device designed and sized for attaching a damaged tendon or ligament to a bone surface, comprising a cylindrical membrane made of a biological material which can promote cell growth, having a first end which is open, and a second end which is closed, and which has a diameter at the closed end which is suited for receiving and holding a bone dowel that has been removed from a large bone via a trocar, for anchoring said implant device and said bone dowel in a hole that has been drilled into a bone surface at an attachment location where a tendon or ligament needs to be reattached to a bone surface.

2. A surgical implant device designed and sized for attaching a damaged tendon or ligament to a bone surface, comprising at least one skirt component made of a processed collagen mesh, and at least one anchoring sleeve which has a diameter at the closed end which is suited for receiving and holding a bone dowel that has been removed from a large bone via a trocar, for anchoring said anchoring sleeve, holding said bone dowel, in a hole that has been drilled into a bone surface at an attachment location where a tendon or ligament needs to be reattached to a bone surface.
